(19) United States
(12) Patent Application Publication
(10) Pub. No.: US 2009/0054904 A1
(43) Pub. Date: Feb. 26, 2009

(54) Methods and devices for eye surgery

(75) Inventor: Jorgen Holmen, Uppsala (SE)

Correspondence Address: YOUNG & THOMPSON, 209 Madison Street, Suite 500, ALEXANDRIA, VA, 22314, US

(73) Assignee: PHACO TREAT AB, UPPSALA (SE)

(21) Appl. No.: 11/989,009

(22) PCT Filed: Jul. 13, 2006
(86) PCT No.: PCT/SE2006/050259

§ 371 Date: Jan. 18, 2008

Related U.S. Application Data
(60) Provisional application No. 60/699,853, filed on Jul. 18, 2005.

(51) Int. Cl. A61F 9/007

(52) U.S. Cl. 606/107

(57) ABSTRACT

A device for removing undesired tissue such as residual tissue, epithelial cells and/or other undesired material(s) from an inner surface of a lens capsule of an eye, includes an elongated body [2] having a proximal and a distal end and at least one central lumen extending between the ends. A flexible shaving filament (4) is movably provided in the lumen so as to be insertable into the lens capsule. The filament has an overall stiffness such that it will be able to conform to an inner surface of a lens capsule when inserted into the capsule, and also to enable shaving off of material from the inner surface. The invention also relates to a method for removing the undesired tissue is also disclosed.

The present invention relates generally to eye surgery, in particular to prevention of secondary cataract (SC). More precisely the invention relates to a device for efficient removal of residual epithelial cells from the lens capsule in preparation for implanting an intraocular lens (IOL), and to a method for such removal.

### BACKGROUND OF THE INVENTION

The crystalline lens of the human eye is located in the posterior chamber between the posterior iris surface and the vitreous body. It is a biconvex transparent tissue without nerves and blood vessels, weighing approximately 0.2 g. The lens is enveloped in a capsule, a structureless, transparent and elastic membrane bag. Approximately 80 zonular fibers, extending between the capsule and the ciliary body, suspend the lens. The inside of the lens capsule consists of lens epithelial cells and lens fibers. The lens epithelial cells form a monolayer underlying the capsule from the anterior pole to the equator of the lens. These cells continue to undergo cell mitosis throughout life in the area located between the anterior pole and the lens equator. The lens epithelial cells that underwent cell mitosis gradually move toward the lens equator and differentiate into lens fibers. These cells make up the rest of the lens. New layers of fiber cells are constantly formed on top of those previously formed. The older fiber cells become denser and during the 3^{rd} decade of life a hard nucleus is formed in the middle of the human lens, consisting of old dehydrated fiber cells.

A cataract is defined as every form of opacity in the lens or its capsule; the lens becomes cloudy, resulting in a loss of visual ability. A cataract is a painless phenomenon, but decreases the quality of life if the lens is not surgically extracted and replaced by an artificial lens.

When the lens is surgically extracted, an incision is made in the anterior part of the eye, i.e., the cornea or the sclera, and an opening is made in the lens capsule by a procedure called capsulorhexis.

Following capsulorhexis, the lens is removed, and remaining parts of the lens, i.e. lens fibers and lens epithelial cells, are removed using an irrigation and aspiration device. After complete removal of the lens, an artificial lens is implanted into the lens capsule or molded inside the capsule.

After cataract surgery, the most common postoperative complication is secondary cataract, or so called anterior capsule opacification/posterior capsule opacification (ACO/PCO), which has the clinical and economic significance to be considered as an important public health problem. Studies report that the incidence of PCO is ranging from 10% to 50% approximately 4 years after surgery. Migration and proliferation of remaining lens epithelial cells is the main cause of PCO. These cells grow from the peripheral parts of the capsule onto the posterior capsule and continue toward the axial region. Impaired visual acuity is the result caused by cell migration, proliferation and aggregation, the production of extracellular matrix, fibrosis and wrinkling of the lens capsule. YAG-laser capsulotomy is the standard surgical procedure to remove PCO.

From an economic point of view, symptomatic treatment of PCO is ranked one of the highest of the medical costs in the U.S.A. Thus, development of a procedure to prevent PCO reduces the medical costs related to YAG laser capsulotomy, including the costs for the treatment, its complications, and YAG laser equipment. Accordingly, there is a great need for PCO prophylaxis.

Mechanical and pharmaceutical methods for PCO prophylaxis by removing or destroying residual lens epithelial cells have been developed. However, none of them has been proved to be practical, effective, and safe enough for routine clinical practice.

ACO relates to the new types of accommodative IOLs whereas the surgery uses a smaller capsulorhexis, sometimes made in the periphery of the capsule, leading to growth of lens epithelial cells onto the anterior capsule, i.e. causing ACO.

Capsular polishing, aspiration of residual lens epithelial cells, ultrasound combined with aspiration, cryocoagulation, and osmolysis are examples of methods that have been developed and shown to remove or destroy remaining lens epithelial cells, but none of these methods have been proven to be enough efficient in PCO prophylaxis.

Related patents are listed below.

U.S. Pat. No. 4,538,611A1 to Kellman, discloses a device equipped with a flange at the front end and a loop to perform lens nucleus cutting. The flange, defined as an essential part of the invention, limits the use in small incision surgery, whereas the corneal incision is about 2 mm or smaller. The patent is silent about using the device for other purposes than lens cutting, and more specifically nothing is disclosed about removing lens epithelial cells from the inner lens capsular surface.

U.S. Pat. No. 4,732,150A1 to Keener, discloses a device for lens nucleus cutting using a snare loop. First, the lens nucleus is displaced into the anterior chamber, then a loop of wire is placed around the nucleus, finally the loop is constricted to divide the nucleus in multiple segments. The patent is silent about using the device for other purposes than lens cutting, and more specifically nothing is disclosed about removing lens epithelial cells from the inner lens capsular surface.

U.S. Pat. No. 4,869,716A1 to Smirmaul, discloses a device and a method for lens nucleus cutting by using a loop. An example is presented comprising a stainless steel wire as loop material with dimension of 0.008-0.015 inches, i.e. 0.20 mm-0.38 mm. The stainless steel wire material of this wire dimension should not be flexible enough to be used for e.g. scraping purposes inside a lens capsule, and furthermore the patent is silent about using the device for other purposes than lens cutting, and more specifically nothing is mentioned about removing lens epithelial cells from the inner lens capsular surface.

WO 9520919A1 to Galan Nieto, discloses a device for lens nucleus cutting by using a “lasso” technique. The patent is silent about using the device for other purposes than lens cutting, and more specifically nothing about removing lens epithelial cells from the inner lens capsular surface.

RU2143223, discloses a device for lens nucleus fragmentation by a cutting snare loop. The patent is silent about using the device for other purposes than lens cutting, and more specifically there is nothing in this patent about removing lens epithelial cells from the inner lens capsular surface.

RU 2190379 to Mamikonyan discloses a device for lens nucleus fragmentation. It comprises a wire loop operable by a spring biased piston. The patent is silent about removing lens epithelial cells from the inner lens capsular surface.

U.S. Pat. No. 6,551,326 to van Heugten et al, discloses a device with a sharp cutting edge at the end of a loop for making circular openings into the lens capsule, i.e. capsulorhexis. The patent is silent about using the device for other purposes, and more specifically nothing about removing lens epithelial cells from the inner lens capsular surface.

RU 2168322 to Kuznetsov, discloses a method and a device for determining the capsular diameter during surgery. The loop is used to determine the capsular diameter. The patent is silent about using the device for other purposes, and more specifically nothing is disclosed about removing lens epithelial cells from the inner lens capsular surface.

RU 2143253 to Andronov et al, discloses a device for fragmentation of the lens nucleus consisting of an operating loop of surgical silk embedded in the surface layers of a silicon loop to catch and split the nucleus. The patent is silent about using the device for other purposes, and more specifically nothing is disclosed about removing lens epithelial cells from the inner lens capsular surface.

U.S. Pat. No. 5,728,117 to Lash, discloses a device including a flexible band with a cutting edge to cut lens capsular tissues to perform a capsulorhexis. The patent is silent about using the device for other purposes, and more specifically nothing is disclosed about removing lens epithelial cells from the inner lens capsular surface.

RU 2029528 to Sheludchenko, discloses a device with a loop for removing a disclocated lens in the vitreous body. The loop is engaged with the lens to withdraw it from the eye, avoiding damage to the corneal endothelium. The patent is silent about using the device for other purposes, and more specifically nothing is disclosed about removing lens epithelial cells from the inner lens capsular surface.

FR 2855746 to Leon, discloses a device including a circular wire with a cutting edge used for cutting a part of a capsule of a lens. The patent is silent about using the device for other purposes, and more specifically nothing is disclosed about removing lens epithelial cells from the inner lens capsular surface.

FR 2855745 to Leon, discloses a device including a ring with a cutting edge for cutting a part of a capsule of a lens. The patent is silent about using the device for other purposes, and more specifically nothing is disclosed about removing lens epithelial cells from the inner lens capsular surface.

CN 1154233 to Wu, discloses a device for lens extraction. The patent is silent about using the device for other purposes, and more specifically nothing is disclosed about removing lens epithelial cells from the inner lens capsular surface.

All these patents are silent about using the methods or the devices for removing or shaving off lens epithelial cells from the lens capsule or capsular polishing, and are not adapted to micro incision cataract surgery (MICS) that is based on corneal incisions of about 1.5 mm or smaller.

There are numerous patents that disclose devices and methods for capsular polishing, but these inventions are very different in structure and operation compared to the method and device according to the present invention. None of these patents relates to a device with a filament or a loop for capsular polishing. Examples of such patents are:
U.S. Pat. No. 6,852,093 to Boukhny (Alcon), discloses a tip for a handpiece to be used for enhanced capsular polishing.
U.S. Pat. No. 6,234,993 to Jordan, discloses a handpiece for removing phaco-emulsified lenses and cleaning the lens capsule,
U.S. Pat. No. 5,814,010 to Ziegler (Allergan), discloses a handpiece tip for irrigation and aspiration with specific arranged vacuum ports in a spaced apart angular relationship with each other.

### SUMMARY OF THE INVENTION

In view of the disadvantages and shortcomings of known methods for avoiding PCO, and for the developing problem with ACO for new accommodative IOLs it would be highly desirable to have access to means and methods for preventing PCO and ACO by efficient removal of epithelial cells and residual lens fibres. It would also be desirable to provide a device with the potential to replace the step of hydrodissection or viscodissection for the separation of the lens from the lens capsule.

Therefore, it is an object of the present invention to provide such means and methods. The object is achieved with a device as claimed in claim 1 and a method as claimed in claim 27.

Thus, a device is provided comprising an elongated body having a proximal and a distal end and at least one central lumen extending between said ends. In said lumen there is movably provided a flexible shaving filament. The flexible shaving filament is made of a suitable material, such as preferably a polymer material, e.g. polypropylene or nylon, although other materials are possible.

The method according to the invention comprises the insertion into the lens capsule of a filament that will form one or more loops inside the capsule. During insertion/removal of the filament and formation of loops, the filament is moved repeatedly back and forth, whereby material is shaved off from the capsule interior wall. The procedure is preferred to be performed after a capsulorhexis has been made, and before the lens has been separated from the capsule by other means, for a convenient integration in the lens extraction surgery. However, it is still possible to successfully use the device and method even when the lens has been separated from the capsule, and/or when the lens has been removed from the capsule. The method and device could also be used to separate an implanted artificial IOL from the capsule and loose any remnants from the inner capsule interior wall.

Further scope of applicability of the present invention will become apparent from the detailed description given hereinafter and the accompanying drawings which are given by way of illustration only. It should be noted that the tolerances and proportions between the filament, piston, hollow tube and tip portion are not accurate in the illustrations, due to large difference in scale between length and width of the devices components, e.g. a length of approximately 100 to 200 mm and a width of approximately 0.3 to 5 mm. The illustrations are thus not to be considered limiting on the present invention. In the drawings

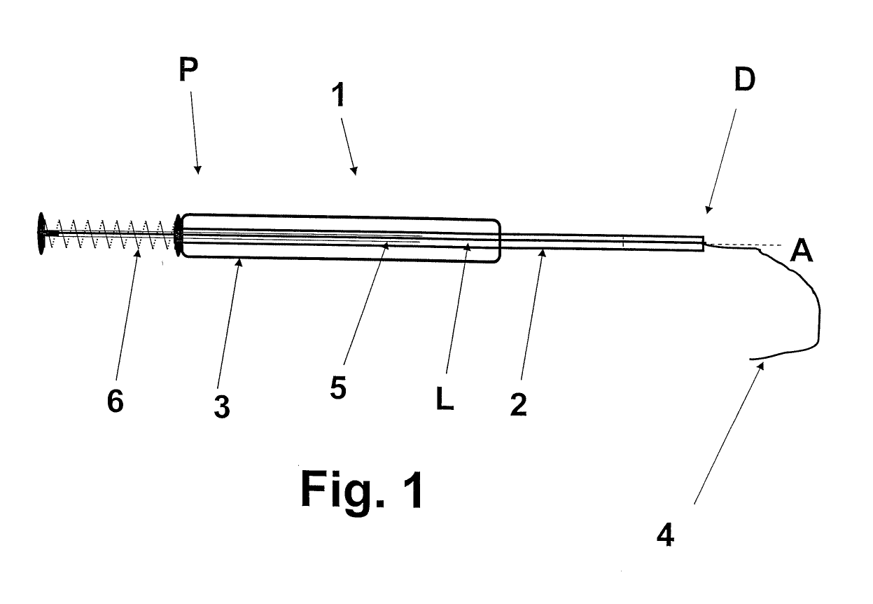

FIG. 1 shows a first embodiment of a device according to the invention including a non-fixed distal end of the filament;

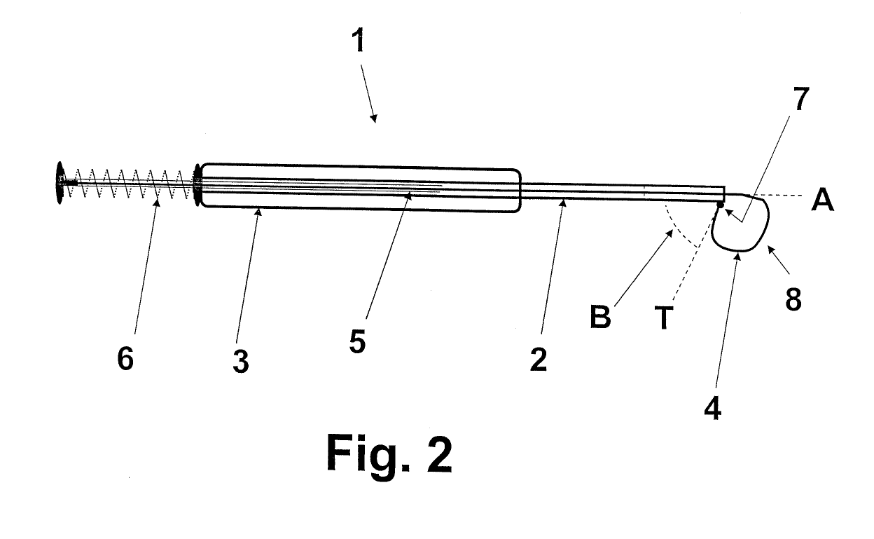

FIG. 2 shows a second embodiment of the device according to the invention including a fixed distal end of the filament;

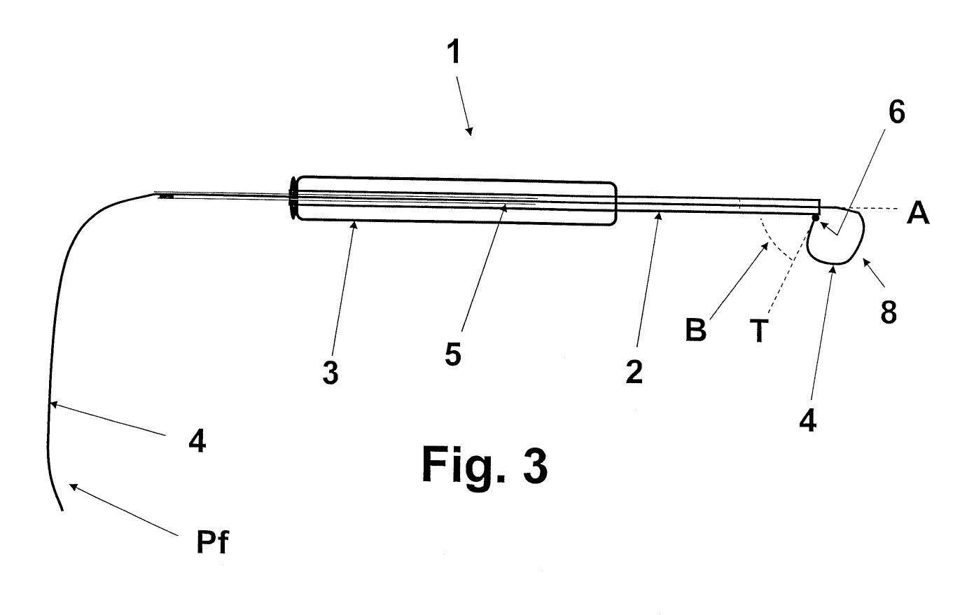

FIG. 3. shows a third embodiment of the of the device according to the invention, including a non-fixed proximal end of the filament.

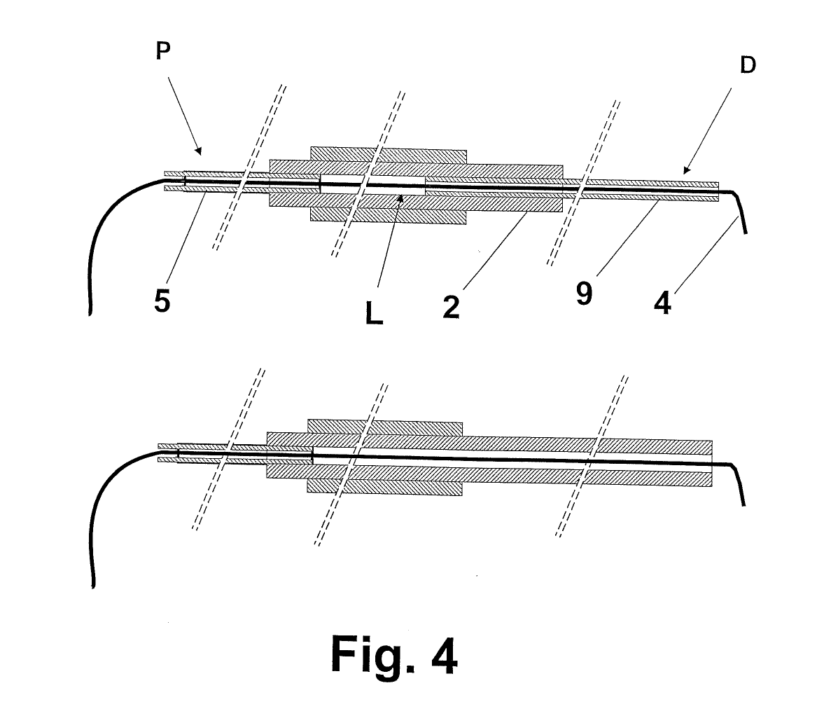

FIG. 4 shows a forth embodiment of the device according to the invention, including a tip portion.

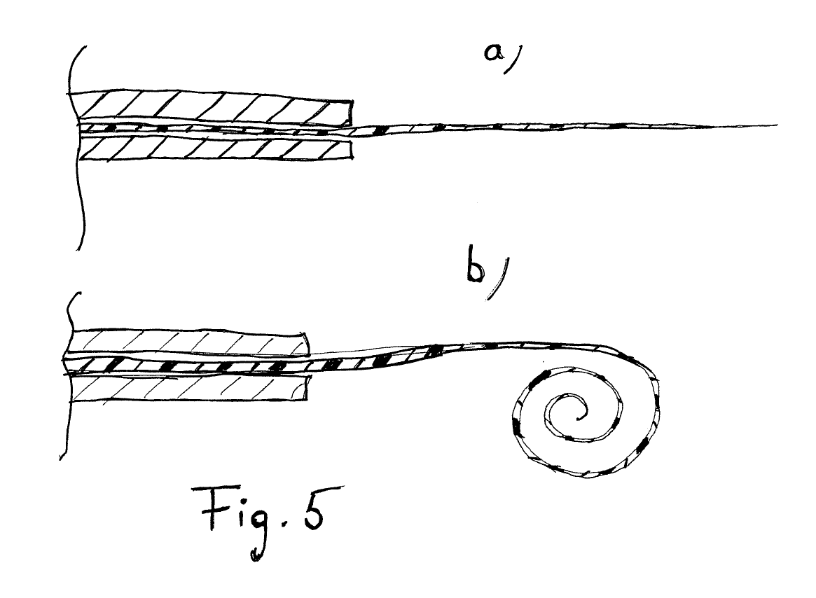

FIG. 5 a and b shows embodiments of the filament and different structures of a non-fixed distal end of the filament;

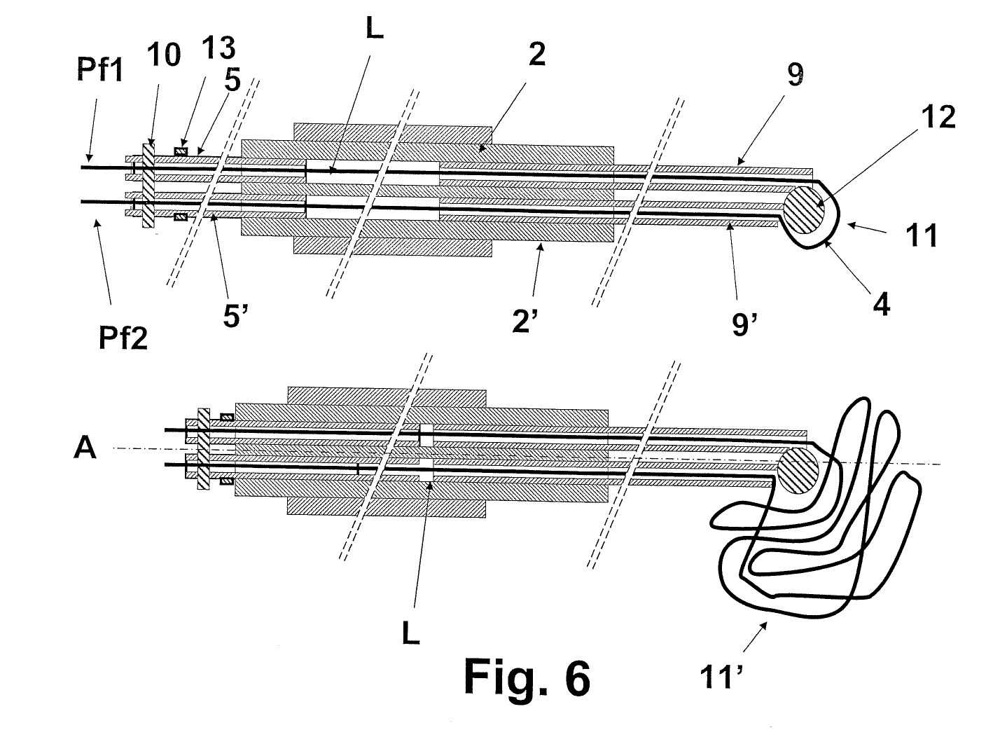

FIG. 6 shows a dual parallel mechanisms for feeding the filament back and forth;

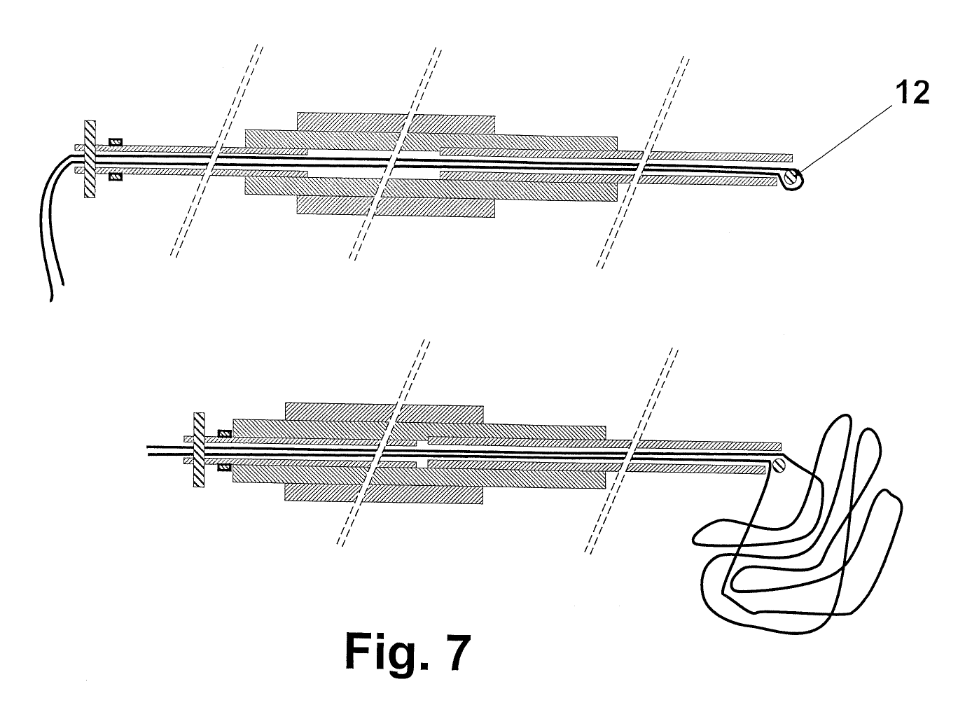

FIG. 7 shows completely fused parallel mechanisms for feeding the filament back and forth;

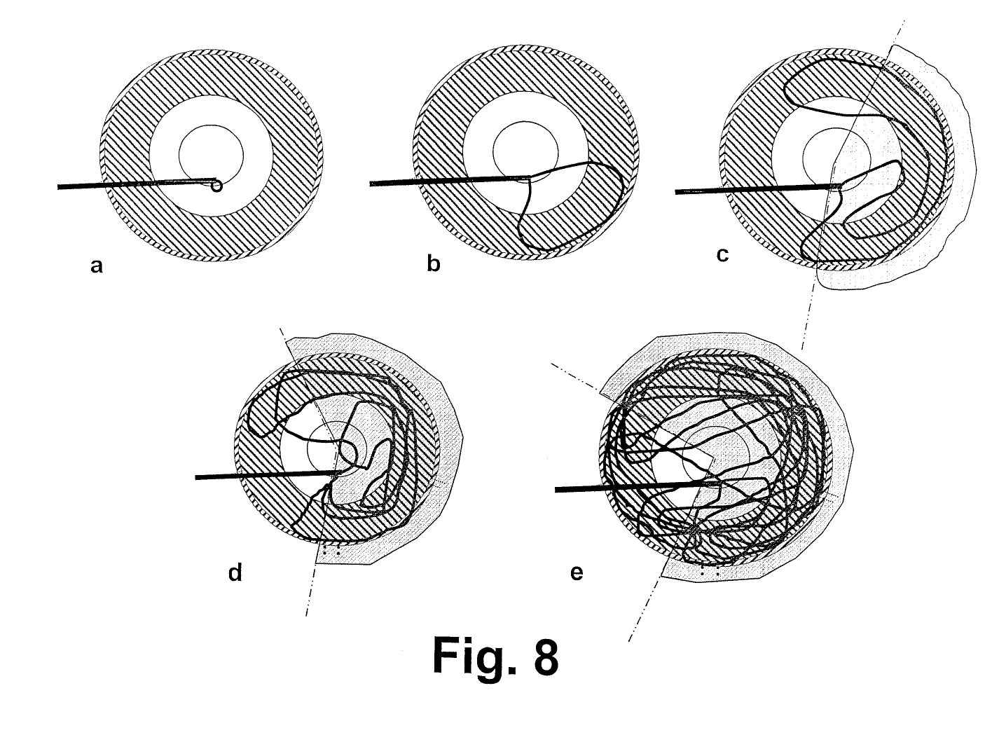

FIG. 8a-e shows the use of one embodiment in surgery, i.e. introducing the device in the lens capsule and forming a loop, a couple of loops, a coil of several loops, and a coil of multiple loops along the lens capsular surface, also showing the effective angles whereas the effect of the shaving off are most efficient;

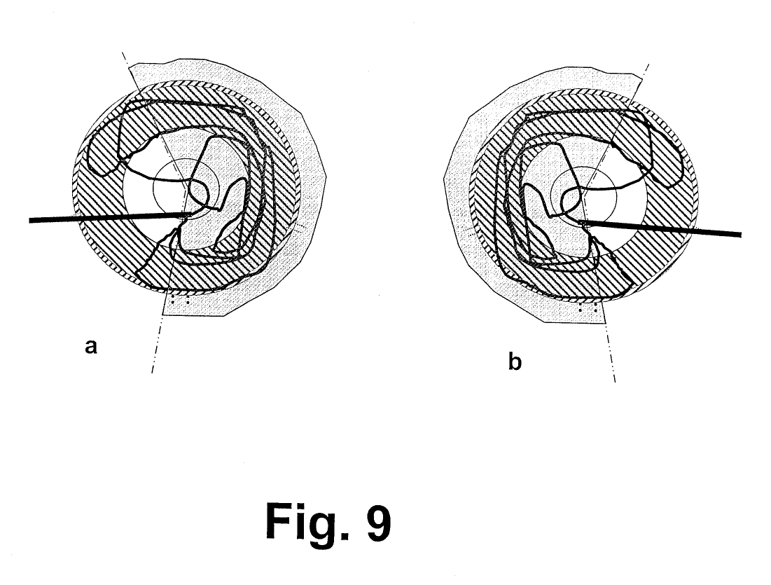

FIG. 9a-b shows the surgery of using one embodiment of the invention from different surgical incisions to cover the complete inner capsular surface.

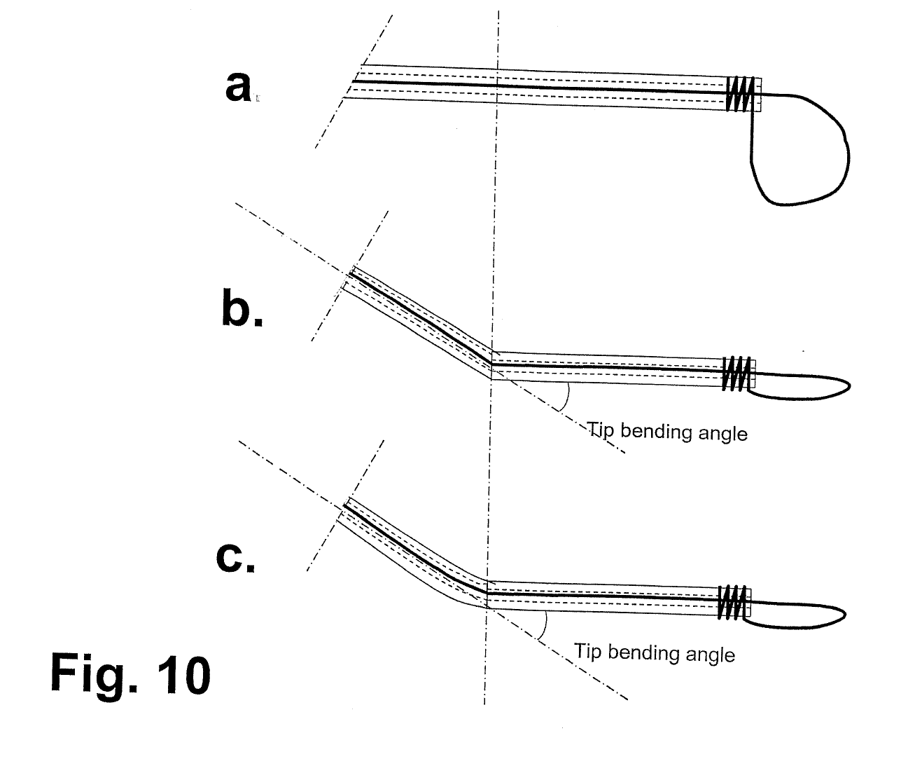

FIG. 10 a-c shows an embodiment of a bent tip, and the position of the bending in relation to a fixated loop.

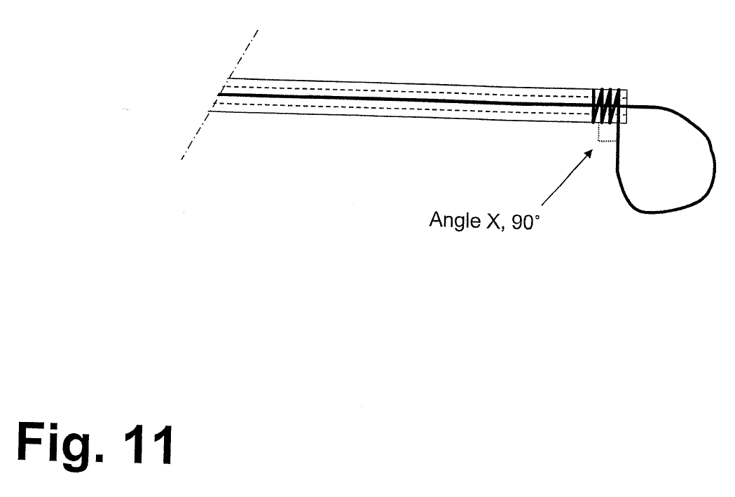

FIG. 11 shows a preferred principle of fixation of the filament to the tip by winding of the filament to the tip, and adding a glue, such as UV-light hardening glue.

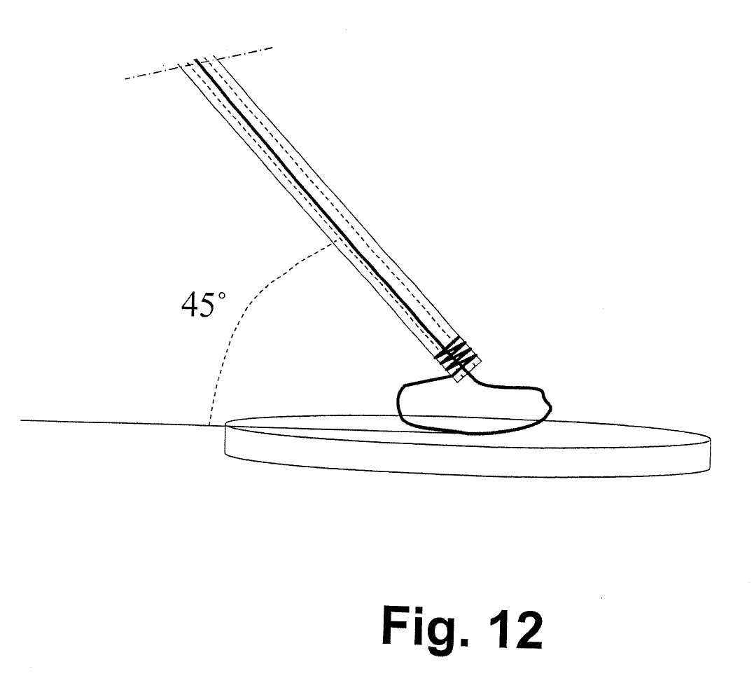

FIG. 12 shows the position of the tip and the loop during a test of filament stiffness/flexibility by exerting a force onto a scale.

### DETAILED DESCRIPTION OF PREFERRED EMBODIMENTS

The present inventor has surprisingly discovered that it is possible to shave off material from the inner surface of a lens capsule by employing a thin flexible shaving filament that is introduced into the capsule. Thus, the thin flexible shaving filament will in this application be referred to as a “shaving filament”.

For the purpose of this application the term “shaving” shall be taken to mean an action of closely cutting off or away epithelial cells or other unwanted tissue or structure from the inner surface of a lens capsule.

For the purpose of this application and invention, a “filament” is taken to mean an elongated solid body with suitable stiffness and flexibility, with a diameter between 0.02 mm and 0.50 mm, preferably between 0.08 mm and 0.15 mm, even more preferably between 0.10 mm and 0.15 mm and made of a flexible biocompatible material.

By the term “biocompatible material” we mean a material that per se is non-harmless to living tissue in a short perspective, i.e. during use thereof as per the present invention.

A suitable stiffness would correspond to a Young's modulus of more than 0.5 GPa, preferably more than 1 GPa, still more preferable greater than 2 GPa, but less than 200 GPA, more preferred less than 100 GPa, and even more preferred less than 30 GPa, and suitable less than 15 GPa.

For the purpose of the present application, “stiffness” (S) of a filament shall be taken to mean a force measured by the method described below.

S=Exerted force (Newton; N)=The reading result×9.81 m/s²

The suitable stiffess/flexibility was surprisingly revealed in an experiment. Loops of different materials, different diameters, and a circumference of 2 cm were included in the test.

Each loop, attached to a rod (the test configuration being a device as disclosed herein), was pressed down on a scale to the half of the diameter of the loop, as shown in FIG. 12. The rod (tip) was held in an angle of 45 degrees in relation to the horizontal plane of the scale plate, and the loop pointing down. The reading of the scale was recalculated to the manual force exerted on the scale. Differences in the weights of the different filaments was estimated to be negligible. Each loop was tested repeated times, and the mean of these readings was calculated, results are shown in Table 1.

TABLE 1
Examples of Stiffness (S) (in Newton) for different filaments formed
as 2 cm loops according to the test method disclosed herein.

| | | Stiffness | |
|---|---|---|---|
| Material | Diameter | (mean ± standard dev) | Fabric |
| Polyamide | 0.12 mm | 13 ± 3 N | Fishing line |
| Polyamide | 0.13 mm | 8 ± 1 N | Suture 5/0 |
| Polyamide | 0.15 mm | 16 ± 3 N | Fishing line |
| Polyamide | 0.16 mm | 23 ± 6 N | Suture 4/0 |
| Polyamide | 0.20 mm | 63 ± 13 N | Suture 3/0 |
| Polypropylene | 0.09 mm | 6 ± 1 N | Suture 6/0 |
| Polypropylene | 0.10 mm | 12 ± 3 N | Suture 6/0 |
| Polypropylene | 0.15 mm | 28 ± 5 N | Suture 5/0 |
| Polypropylene | 0.25 mm | 126 ± 22 N | Suture 3/0 |
| Stainless Steel | 0.12 mm | 216 ± 48 N | — |

Of the above sample filaments the stainless steel was not operable according to the present invention, whereas the other were

Thus, according to the test method mentioned above and designed for the purpose of this invention, a suitable stiffness would thus correspond to a force exerted on a scale of more than 1 Newton, preferably more than 3 Newton, still more preferable greater than 4 Newton, and suitable more than 6 Newton, but less than 140 Newton, more preferred less than 70 Newton, and even more preferred less than 35 Newton, and suitable less than 20 Newton. Note, the standard deviation should be considered, as illustrated in the examples below.

However, stiffness is not the only parameter that can be used to define a suitable filament. Another parameter is flexibility. For the purpose of this application the term “flexibility” of a filament shall be taken to mean the ability of a filament to conform to new shapes without permanent deformation, and allow to retain the old shape again. The flexibility of the filament should be as high as possible, and be able to form one or several new loops with suitable diameter and shape inside the lens capsule, after it has been retracted to a very small loop during insertion through the corneal incision and the capsulorhexis, such as having a loop diameter of 2 mm or less, preferably less than 0.5 mm, and most preferably a completely constricted loop, i.e. a loop diameter of 0 mm.

Another parameter is endurance/tenacity, i.e. the power to maintain the stiffness and flexibility over time and during the performance. High endurance is a good quality for the filaments, so that the device may be used extensively during surgery. For example, a reasonable maintained flexibility and/or stiffness for repeated use through several incisions during a surgical use time of at least 3 minutes, and preferably at least 10 minutes, including repeated constrictions of the loop.

If the filament has an appropriate flexibility and stiffness, it will conform to the inner surface of the capsule, thereby forming loops, one after the other, as more of the filament is fed into the space inside the capsule, finally resulting in a coil of multiple loops. If the filament is moved back and forth, i.e. made to “oscillate”, simultaneously with the feeding of the filament, or intermittently during short pauses in the feeding of the filament, or simultaneously with the pulling out of the filament, a very efficient shaving off of material (residual epithelial cells and lens fibres and/or artificially implanted material such as an IOL) on the inner surface of the lens capsule is achieved. Preferably, a length of filament that is sufficient for forming at least one loop, and suitably more, such as two to twenty loops or even more, will be introduced into the lens capsule. Every loop formed will contribute to an efficient removal of material from the inner capsular surface.

The actual length of introduced filament in a fully projected state inside a lens capsule should amount to at least 1 cm, preferably more than 2 cm, more preferably to more than 2.5 cm, even better more than 3 cm, but preferably less than 100 cm, more preferably less than 50 cm, preferably less than 35 cm, and suitably less than 25 cm.

For embodiments, wherein a single loop is formed, it is preferred that the filament is between 1 cm and 6 cm, preferably more than 2 cm, or even better more than 3 cm, but preferably less than 5 cm, and suitably less than 4.5 cm. An optimal length would be 3 to 4 cm.

For embodiments with multiple loops, the actual length of introduced filament in a fully projected state inside a lens capsule could amount to between 5 cm and 50 cm, or even up to 100 cm, preferably more than 10 cm, but preferably less than 35 cm, and suitably less than 25 cm. An optimal length would be 15 to 20 cm. A relationship exist between the filament diameter and the amount of filament that could be introduced in the capsule, i.e. a small diameter, such as 0.12 mm, or even less, makes it possible to introduce more of the filament in the lens capsule.

The filament could be coloured to enhance visibility of the filament movements in the lens capsule. Similar dyes used for suture filaments should be suitable.

The following parameters are critical to the present invention:

### Relationship Between the Diameter and the Material of the Filament

The choice of diameter is depending of the choice of material to achieve the suitable flexibility and stiffness. Thus, flexibility and stiffess are related to both the dimension and the material of the filament. Generally, the diameter effects are much more pronounced than the material stiffness effects. A test have shown that increasing the inherent stiffness of the material by 10 percent increases the filament stiffness by 10 percent. Increasing the diameter by 10 percent increases the filament stiffness by 46 percent. For example, fluorocarbon is about 30% stiffer than nylon on average, which would mean that the diameter of the filament could be slightly smaller when made of fluorocarbon, as described by Professor Graig Spolek at the Mechanical and Materials Engineering Department, Portland State University, Oregon. (references: Proceedings of the Society of Experimental Mechanics Annual Conference on Experimental and Applied Mechanics, pp. 498-501, 2001; Fluorocarbon Tippet Versus Monofilament, July 2003 Fly Fisherman; http://flyfisherman.com/skills/gstippetsiffness/)

### The Material of the Filament/Wire to Form the Loop

Examples of suitable filaments and materials for the present invention are either a monofilament, a pseudomonofilament or a multifilament, and coated or uncoated, polymers, allays and/or composites, similar to known suture materials and fishing lines. The filament should preferable be non-absorbable, but not excluding the possibility to use absorbable suture materials as well. Examples of materials are plastic, nylon (Ethilon®, Dermalon®), polypropylene (Prolene®, Premilene®), Polyamide (Dafilon®, Supramid®), polyester (Miralene®), polyvinylidine fluoride (fluorocarbon), hexafluoropropylene-VDF (Pronova Poly®), silk or other biocompatible materials. Filaments could be coated with polytetrafluoroethylene, PTFE (Teflon®), silicon (PremiCron®), silicone combined with PTFE, polyethylene vinyl acetate, PEVA (Synthofil®) or similar biocompatible coatings. Absorbable suture materials would also be an option, but of less added value since the filament should be removed, such as polygalactin 910 (VICRYL®), poliglecaprone 25 (MONOCRYL®), polydioxanone (PDS II®) and surgical gut. Another option would be to coat and/or soak the filament with an active agent for local treatment of lens epithelial cells, such as 5-fluorouracil or specific substances directed to these cells.

Prior art patents refer to use a wire for a loop for lens fragmentation, since a cataractous lens nucleus can be very hard. A fragmentation would require a wire that in a proper sense is of metal, with properties aimed on strength and cutting, such as possessing high traction force capability, rather than the parameters of the present invention.

### Criteria of Filament and Device

The proportion of the device between the inner diameter of the cylinder where the filament is running and the diameter of the filament is critical. This is of less importance when the loop is a wire, that in a proper sense is of metal, because of the material stiffness, i.e. having a high Young's Modulus, such as 100 GPa or more, in combination with a high filament diameter, such as 0.30 mm or more. The proportion is however very essential to make it possible to move a flexible filament with a small diameter, such as 0.20 mm or smaller, in combination with a low Young's Modulus, such as 30 GPa or lower, forward without entanglement inside the cylinder of the device when the movement of the filament along the capsular surface meet resistance. The proportion shown in the illustrations in the prior art patents, referred to in the Background section, i.e. large cylinder volumes in relation to a thin wire, would most likely make the instrument very inefficient with a high risk of entanglement.

### The Length of the Filament to Form the Loop/Size of Loop

The lens capsule is approximately 22.3 mm in circumference, estimated by using the following formula derived by the Indian mathematician S. Ramanujan (1914) for estimating the circumference of an ellipse, whereas the mean diameter (2a) of the human crystalline lens is about 9.6 mm and the thickness (2b) about 4.1 mm.:

$$P \approx π ( a + b ) [ 1 + 3 h / ( 10 + \sqrt{4 - 3 h )} ]$$

wherein h=(a−b)²/(a+b)²
wherein a is the major radius, and b is the minor radius, i.e. a>b.

The length of the introduced wire to perform fragmentation of a lens nucleus or measurement of the capsule inner diameter would typically be about 25 mm, i.e. enough to embrace the lens nucleus or to measure the capsular diameter. The prior art patents referred to in the Background section are silent about including a longer wire to accomplish the desired purposes.

### Fixation Angle of the Filament to the Tube or Tip Portion

The angle between the filament and the central axis of the tube or tip portion at the distal fixation point is an important parameter. The prior art patents have not considered this.

Now the device according to the invention will be described with reference to the drawing figures.

FIG. 1 shows a schematic representation of a device according to the present invention in a first embodiment.

The device, generally designated with reference numeral 1, comprises a tube 2, suitably made of plastic or metal such as steel, and having a proximal (P) and a distal (D) end and a lumen (L). Preferably, but not necessarily, there is provided a gripping member 3 on the tube to facilitate handling of the device. This gripping member can be made of a polymer material, such as a piece of tubing fitting snugly on the hollow metal tube 2. The gripping member can be surface structured for enhancing the handling properties. The gripping member and the hollow tube could be made in one piece and be the same unit.

In the lumen L of the hollow tube 2 there is provided a thin flexible filament 4, having a proximal and a distal end. The filament has certain properties (to be described below) that enables the filament to act as a shaving device when brought into contact with the inner surface of a lens capsule.

There is also preferably provided means for feeding the flexible filament 4 out of the distal end of the tube 2, such that the filament will project out further and further from the tube. This means is suitably a mechanism for facilitating the feeding of the filament into the lens capsule.

In one embodiment this mechanism comprises a piston 5, with a proximal and distal end and a lumen, arranged inside the hollow tube 2, and in which the filament 4 runs.

In one embodiment the proximal end of the filament 4 is fixed to the piston 5, preferably in the proximal end of the piston 5, e.g. by glue or clipped to said end. Thereby, the movements of the filament and the piston is limited, and only a fixed length of filament can be introduced in the lens capsule. The part of the filament to be introduced in the lens capsule should not exceed the length of the piston. Thereby the piston can be prevented to be removed from the hollow tube by accident if the distal end of the filament 4 is fixed to the tube 2.

In one embodiment, similar to the previous one, the piston 5 is forced backwards by means of a spring 6 or equivalent, thereby facilitating the repeated movements of the filament 4 back and forth.

In a further embodiment of the device according to the present invention, and in a set up of a single mechanism, i.e. one piston, one hollow tube with or without the tip portion, the filament is fixedly attached to the hollow tube at the distal end, or said tip portion when that is included. This embodiment is schematically illustrated in FIG. 2.

The embodiment of FIG. 2 exhibits the same basic structure and components as the embodiment shown in FIG. 1, but the filament 4 is attached by glue or some other suitable fastening means 7 on the tube 2 at the very distal tip thereof so as to form a loop 8. The filament 4 forms a specific angle B with the central axis A of the tube 2 at the point of attachment, the angle being defined as follows:

An angle B of 0 degrees means that the tangent (indicated with reference numeral 1) of the loop of the attached filament is pointing straight backwards parallel to the shaft, and 180 degrees means that the attached filament is pointing straight forward parallel to central axis A of the shaft. The angle B can be between 0 and 180 degrees. The angle B is of importance for the formation of the loop. An attachment at 180 degrees leads to a loop pointing straight forward, and make it possible to introduce the device through very small incisions, such as less than 1.0 mm, because the loop in contracted form is straight in front of the tip. An attachment at 0 degrees creates a loop that points in about 90 degrees in relation to the central axis A or more from the tip, which makes the device more suitable to reach areas to the sides, which is a very desired ability, however the price for that is that the instrument will need a larger incision than in previous option. This leads to a suitable angle B of between 90 to 160 degrees, preferably about 115 to 135 degrees, i.e. the device reaches areas to the sides in the capsule and is still small enough to be introduced through a small incision. However, angles out of this preferred range could also be of value for specific abilities. A suitable angle B has in different tests in animal eyes been found to be 90 degrees, and in a wider definition to be about 90±30 degrees.

In one embodiment shown in FIG. 3, the proximal end Pf of the filament 4 is not fixed to the piston 5. Thereby, an extensive length of filament 4 can be introduced in the capsule, i.e. much longer than the length of the piston 5, as described in the previous two embodiments. The piston 5 carries out the feeding of the filament 4 into or out of the capsule. It is achieved by successively moving the piston back and forth, whereas the filament 4 is manually and temporary fixed to the piston by the surgeon's fingers either at the backward movements or the forward movements depending on which direction the surgeon wants to feed the filament. The same principle is used to “oscillate” the filament by repeated movements back and forth, and meanwhile holding the filament 2 fixed to the piston 5.

In one embodiment a temporary fixation of the filament 4 to the piston 5, is executed by a mechanism instead of manually, as described in previous embodiment. When the surgeon push the piston 5 forward, a couple of jaws softly grab the filament simultaneously, allowing the filament 4 to be moved forward and backward as long as the jaws hold the filament. When the piston 5 reaches the end position the jaws releases the filament 4. Then, the piston 5 is moved backwards, preferably driven by a spring or equivalent, during which the jaws is in a released position and the filament 4 unfixed. This is achieved by for example a steering-gear mechanism with a pin running in a control track, e.g. one track for feeding the filament 4 back or forth, and one track for moving the piston 5 but not the filament 4. The surgeon can then choose to the feed the filament 4 or to move the piston 5 backward or forward to go for a new grab without moving the filament 4. This choice is achieved either by turning the piston 5 within the tube 2 or accomplished by an automatic switch at the end of the control tracks, similar to the mechanism of a ball pen, optionally moving and fixating the ink cartridge and the point of the ball pen. The skilled person would be able to design and adapt other equivalent mechanisms for the same purpose without inventive work or undue experimentation.

For example, a soft and flexible tube made of silicone or equivalent material can be fixed on the proximal end of the piston, and the filament can be temporarily manually fixed by the surgeon, by squeezing the tube together manually by the fingertips. Another way is to have an extended slit opening along the proximal part of the piston of about 5-10 mm, or similar extended slit opening on the tube attached to the distal end of the piston. The filament running inside the piston and/or tube may be temporarily fixed manually by the surgeon, by pressing a finger towards the extended slit opening, where the filament is exposed.

It is important that there is a very close tolerance between the filament and the piston lumen. If there is too much space available inside the piston, the filament will tend to flex therein, and such flexing may give rise to the occurrence of kinks on the filament. Preferably the inner diameter of the piston is less than 0.20 mm larger than the filament diameter, more preferably less than 0.10 mm larger, and most preferred less than 0.05 mm larger than the filament diameter, i.e. as small difference as possible. However, considerations of the tolerance must be made in respect of the degree of variations in the diameter, memorized curvature of the filament diameter, as well as potential swelling of the filament due to a potential absorption of water or change in temperature.

This holds true also for the tolerance between the filament and the lumen of the tube 2. However, here a slight less close tolerance can be accepted to allow the movements of the piston placed between the filament and the tube 2. Thus, the inner diameter of the hollow tube should be less than 1.0 mm larger than the filament diameter, preferably less than 0.50 mm larger and most preferred less than 0.40 mm larger than the filament diameter, with still enough space available to embrace the hollow piston.

If the piston 5 is too thick-walled, the lumen in the hollow tube 2 must have a correspondingly larger inner diameter. This could cause the same problems of flexing of the filament 2 in the region of the hollow tube 2 from the distal end of the piston 5 up to the distal end of the hollow tube 2. Thus, it is desirable that the piston 5 is made of a piece of a very thin-walled tube, which must be rigid enough to allow it to act as a piston. The inner diameter of the hollow tube 2 should be less than 0.05 mm larger than the outer diameter of the piston 5, preferably less than 0.02 mm larger and more preferred less than 0.01 mm larger. Most preferably the inner diameter of the hollow tube should be as small as possible, but no more than until a slight resistance is achieved when moving the piston 5 inside the hollow tube 2. Thereby, there is very little space left over for the filament to flex when it is forced forward within the hollow tube. Thus, the filament is kept straight and can be moved forward with force enough to shave the inner surface of the capsular wall at which the filament will meet some resistance.

In order to alleviate the problem of potentially occurring kinks on the filament, there can be provided a tip portion 9, shown in FIG. 4, inserted in the hollow tube 2. The tip portion 9 is in itself a piece of tubing having a lumen similar in size to the lumen of the piston 5. In this way the tendency of the filament to flex and form kinks is greatly reduced, if not eliminated. The inner diameter of the tip portion should be less than 0.50 mm larger than the filament diameter.

Thus, in one embodiment, shown in FIG. 4, the length of the lumen L between the filament 4 and the hollow tube 2 is minimized by shortening the hollow tube to a length about the same as the piston 5. Then, a tip portion 9 with a proximal and a distal end is fixed with its proximal end at the distal end of the hollow tube 2. The tip 9 and the piston 5 could be made with similar dimensions, i.e. similar tolerance as for the tip 9, as for the piston 5 and the filament 4 and between the piston 5 and the hollow tube 2 as described previously. However, the tolerance between the tip 9 and the hollow tube 2 should preferably be very tight, and preferably fixed, glued or clipped together. Another advantage of the tip portion 9 is that the dimension can be very small, such as between 0.2 and 0.6 mm, to allow use of the device through micro incisions, i.e. incisions smaller than 1.5 mm.

In one embodiment in which the tip 9 described above is used, the length of the lumen L of the hollow tube 2, measured from the proximal end of the tube to the proximal end of the tip 9, is shorter than the length of the piston 5. Thus, the piston 5 is prevented from being moved too much into the hollow tube 2. Otherwise, the piston 5 would completely disappear within the hollow tube 2 and very difficult to withdraw without dismounting the device.

In another embodiment, the length of the lumen L of the hollow tube 2, measured from the proximal end of the tube to the proximal end of the tip 9, is longer than the length of the piston 5. To prevent the incidence of a completely introduced and disappeared piston 5 within the hollow tube 2, as described as a potential dilemma in the previous embodiment, the piston 5 is provided with a stopper 13 at the proximal end, as shown in FIG. 6. The stopper 13 could be either a bending of the piston, an enlarged diameter of the piston, a globe or other kind of material fixed to the proximal end that prevent the piston from disappearing inside the hollow tube. The positive result is that the potential risk of crushing the filament between the tip 9 and the piston is minimized. However, the lumen L between the distal end of the piston, completely introduced to the stop, and the proximal end of the tip 9 should be as small as possible, such as 5 to 10 mm, but preferably not smaller than keeping a safety distance of about 1 to 3 mm, preferably about 5 mm, as illustrated in FIG. 6.

A simpler embodiment for the feeding is simply achieved by manually pushing the filament at the distal end of the tube 2, by gripping the filament at a short distance from the proximal end, and forcing it into the tube, whereby it will extend out a corresponding length at the distal end. This procedure is repeated until a sufficient length of filament has been introduced into the lens capsule. During the process of introducing the filament, the operator can push and pull the filament rapidly to cause an oscillating movement of the filament inside the lens capsule, a movement that will bring about an efficient shaving off of material from the inner surface of the lens capsule.

It should be noted that the friction between the filament 4 to the inner wall of the other hollow elements, i.e. the inner lumen of the piston 5, the hollow tube 2, and the tip 9 must not be too high, because when the piston 5 has moved one stroke, thereby having fed the filament 4 into the lens capsule, it must be possible to retract the piston while at the same time the filament must remain inside the lens capsule and not withdrawn together with the piston 5. Friction between the said components also negatively influence the forward movement of the filament 4 into the capsule and should be kept at minimum to prevent the filament 4 from flexing.

The friction between different parts of the filament 4 at crossings between two loops inside the lens capsule should also be kept as small as possible, to avoid entanglement inside the lens capsule.

However, in one embodiment of the device the surface of the filament 4 is changed to exhibit increased friction between the inner capsular surface and the filament to enhance the capability of loosening of material from the lens capsular surface. Such modulation of the surface of the filament 4 should be made only to the extent so that the previously mentioned dilemmas is still avoided, i.e. reduced capability of moving the filament forward due to friction between the filament and the inner walls of the piston, tube and tip, as well as potential risk of entanglement due to friction between different parts of the filament at loop crossings.

Obviously there must be provided some way to lock the filament to the piston during the forward feeding motion, and to release the lock when the piston is retracted. The simplest way to achieve this is by the operator pinching the filament and the piston at the proximal entrance of the piston such that he is able to push the piston and the filament in unison into the hollow tube 2. When the piston is retracted, the pinch on the filament is released, and the filament will not move back together with the piston. This may however not be an optimal solution, and suitably there is provided a mechanism that automatically will achieve this pinching and releasing action. There are numerous technical solutions for this, and the skilled man would be able to devise a suitable mechanism without inventive work.

As indicated above, the general inventive concept can be seen in the provision of a shaving filament 4, which during its feeding into the lens capsule will shave off residual epithelial cells and lens fibres from the inner surface of the lens capsule. In the embodiment described above, the filament has a free end which will conform to the inner surface and form loops as more of the filament is introduced.

The very tip of the filament is preferably designed in a special way, illustrated in FIGS. 5a and b. Namely, in order to avoid a potential problem of the filament tip either finding its way into some wrinkle or even into the incision through which it is introduced, or even worse, penetrating the lens capsule, in both cases resulting in the filament exiting from the capsular interior, the tip is modified to comprise a means for preventing such events to happen. Such means can take various shapes and forms, a few of which will be explained in some detail below.

In one embodiment the distal end of the filament 4 can be tapered, i.e. made thinner and thinner such that it over an end region of say 0.5 to 3 cm gradually decreases its diameter (see FIG. 5a), and consequently also the stiffness will decrease down to essentially 0 N at the very tip of the filament. An other alternative is to let the material change its characteristics so as to be softened. Thereby the tip of the filament 4 will be extremely flexible and will form a very loose end that certainly not will have the capability of penetrating the tissue, and most unlikely will find its way out through the incision. Another alternative is to modify the surface of the distal end of the filament 4 to achieve a high friction between the distal end of the filament 4 and the inner capsular surface. Thereby the filament 4 is prevented from moving forward and slip out of the capsule. Any or all combinations of these options could be used to achieve good control of the tip of the filament 4.

In another embodiment (see FIG. 5b) the tip of the filament 4 is provided with a spiral structure, i.e. it has been pre-shaped to a spiral, and by virtue of its inherent resilience/flexibility it will regain the spiral structure. Such a spiral will be bulky and will efficiently prevent the filament from escaping or penetrating the tissue.

In a still further embodiment the filament end is blunted, meaning that the end is made non-sharp by an enlarged body positioned at the end of the filament, such as a globe, plate, or formation of a closed loop of the filament itself or equivalent structure rendering the end non-sharp.

In the embodiments of FIG. 5, the filament has a free end said free end being insertable into the lens capsule, and wherein the stiffness (S) of the filament varies from between more than 1 Newton, preferably more than 3 Newton, still more preferable greater than 4 Newton, and suitable more than 6 Newton, but less than 140 Newton, more preferred less than 70 Newton, and even more preferred less than 35 Newton, and suitable less than 20 Newton, as measured by the method disclosed in the specification, over the majority of its length, and decreases towards the free end, preferably down to near 0 N at the very distal end of said filament.

Still further variations are conceivable and any structure meeting the object as outlined above is within the scope of the invention.

In one embodiment of the device, schematically illustrated in FIG. 6, the components and the mechanism comprising the piston 5 and tube 2 with or without the tip portion 6 are made in twofold. The following description will include the tip portions, though they could also be excluded, i.e. being replaced by longer tubes 2 and 2′. The two sets are placed parallel to each other, as illustrated in FIG. 6, whereby the tubes 2 and 2′ are fixed to each other, side by side, as well as the optional tip portions 6 and 6′. The pistons 5 and 5′ can be fixed to each other at their proximal ends, using a locking member 10, but this fixation can be temporarily loosened by releasing the locking member 10. Thus, it is possible to freely move one piston independent of the other piston. The filament 4 is passed through both sets of parallel components, i.e. one end of the filament 4 is located at the proximal end of the first piston 5, running through the first hollow tube 2 and the first tip portion 9, forming a loop 11 or a coil 11′ of loops of filament between the first tip portion 9 and second tip portion 9′, then running through second tip portion 9′, the second hollow tube 2′ and finally through the second piston 5′, at which the second proximal end of the filament will be protrude. Thus, the filament has two proximal “loose” ends Pf1, Pf2 and a distal loop or coil portion 11, 11′. Thereby, two separate mechanisms can feed the filament into the lens capsule, combined or individually. This embodiment is advantageous in that it will be possible to feed twice the amount of filament into the capsule in that the filament can be feed into the lens capsule from both its proximal and distal ends simultaneously. The tip portions are designed to guide the filament two “legs” in desired directions, such as in a specific angle B in relation to the central axis A of the device, as well as in a desired angle between the legs. The “legs” of the filament refer to the two sections of the filament that are pointing out of the two tip portions and together form the initial loop. There is also provided a smooth bevelling between the tip portions, illustrated by a rounded deflecting member 12, to avoid damaging the filament, to avoid damaging the filament when it is contracted as a mini loop between the tip portions during introduction and pulling out of the eye. The deflecting member is an option, and the same function could be achieved by shaping the tips appropriately rounded off.

In one embodiment, based on the previous one, the filament 4 is fixed in both pistons in the same way as described for the set of one mechanism. When the pistons are completely pulled backward a mini loop is formed between the two tip portions, and the pistons 5 and 5′ are prevented from being pulled out of the hollow tubes. When introduced with the combined tip portions 9 and 9′ pointing into the lens capsule, eg. between the lens and the capsule, the pistons can be moved forward, thereby feeding filament 4 into the lens capsule at twice the speed, and twice the amount compared to the set of one mechanism. The increased length of introduced filament 4 along the inner capsular surface is significant to increase the efficacy of shaving off material from the capsular wall, by forming a coil 11′ of multiple loops of filament. The ability to temporarily loose the pistons 5 and 5′ from each other is important to enable individual feeding back and forth of the two filament legs, to avoid entanglement between loops at an early stage.

In another embodiment the proximal and distal end of the filament 4 is unfixed in the pistons 5 and 5′, and the mechanisms works as described for the one set mechanisms, but at twice the speed and the possibility to work individually with either leg of the filament. It is of great value to enable removal of the filament 4 by withdrawal from either mechanism, in case of an emerging entanglement in the coil 11′ of loops. The principle of temporarily fixating the filament to the pistons 5 and 5′ for moving the filament 4 back and/or forth works as previously described for the one mechanism, as well as the automatic driven feeding back and forth by the specific steering-gear mechanism.

In one embodiment of the device, shown in FIG. 7, the two dual parallel mechanisms are fused together, e.g. one of the dual components, such as the tip portions, some of the dual components, such as the hollow tubes and the pistons, or all of them. The last of these alternative fusions will be described below, since the other options easily can be figured out by combining the details of the dual parallel mechanisms and the fused mechanisms. The inner diameter of the piston, the hollow tube, and the alternative tip portion is made larger and adapted to hold both “legs” of the filament, preferably with an oval inner lumen, to embrace the filament effectively, and with equivalent considerations of tolerances between the components, i.e. the hollow piston, the hollow tube, the hollow tip portion and the filament, as previously described. One option is to have circular lumens of the piston, hollow tube and tip portion, and instead include a filament with an oval or a half-moon cross-section. The distal end of the tip portion has two specially designed openings with a smooth bevelling between them to prevent the filament from damaging when it is fully constricted for introduction into the lens capsule, as well as guiding the legs of the filament in a desired angle B in relation to the central axis, such as 130 degrees to the left or right in relation to the central axis A. This could be achieved by means of a deflecting member like in the embodiment of FIG. 6. It is also possible to include the different principles of fixed or unfixed filament in the piston, as well as the automatic movement back and forth of the filament. The described construction of a fused mechanism is valuable to keep production costs and amount of components as low as possible.

In a still further embodiment of the device according to the present invention, and in a set up of whereas the filament 4 has a distal end that is reversed as described in several previous embodiments, only one of said ends is being used for feeding the filament 4 into the lens capsule. The other end is in this respect inactive. However, if for some reason the feeding end of the filament 4 gets stuck in the tube 2 or in the lens capsule, or if it for some other reason is not possible to use the feeding end of the filament for retracting the filament, the other “inactive” could be used for that purpose.

The filament 4 itself and its properties requires some consideration. It must be sufficiently flexible to easily form loops 11 once it has been introduced inside the lens capsule, and during the feeding motion thereof. At the same time it must be sufficiently rigid such that the risk of kinks to occur during feeding of the filament 4 is prevented. Furthermore, the diameter of the filament 4 must not be too high since this increases the stiffness very much with less ability to form a coil 11′ of loops and a risk of injuring the capsule. These in some aspects contradictory properties must be appropriately balanced. Suitably the filament diameter is in the range of larger than 0.05 mm, but should not be larger than 0.50 mm, depending on the material properties.

The material of choice for the filament 4 is not strictly critical, and the filament 4 can thus be made of in principle any material that meets the requirements outlined above. At present a filament 4 made of nylon and polypropylene is preferred. Examples of filaments usable in the invention, although the invention is by no means restricted thereto, are various types of fishing-lines made of nylon, and suture materials made of polyamide or polypropylene. These can be made in different ways and are often surface coated, and referred to as tempered monofilament, coated with silicon-PTFE, polytetrafluoroethylene, PTFE (Teflon®)

However, various other types of polymer materials or metals could also be used, such as different kind of suture materials, certain qualities of steel, memory metals, alloys, Nickel-Titanium alloys such as nitinol, alone or in combination with other materials mentioned earlier, e.g. composite metal/polymer materials, such as a core of metal surrounded with a plastic body or other polymer materials, as long as they meet the criteria set forth herein.

For most purposes a filament 4 having a circular cross-section is suitable, but it is possible to employ filaments with more or less elliptical cross-section. It is also conceivable to use a flat ribbon-like filament, in order to provide a still more efficient shaving action.

Now the method according to the invention will be described with reference to FIGS. 8 and 9.

Thus, there is provided a novel method of removing unwanted material such as residual lens epithelial cells, lens fibres, as well as implanted materials such as from an implanted artificial IOL, from the inner surface of a lens capsule of an eye of a mammal. The method is performed after capsulorhexis, but either before or after the lens has been extracted. Thus, there is already an incision made in the capsule which is used for accessing the interior of the lens capsule.

The method comprises introducing a shaving filament 4 (as discussed above) into the lens capsule through said incision (FIG. 8a), either continuously or in small increments. When a length of filament 4 corresponding to the inner circumference of the capsule has been introduced (FIG. 8b), a loop has formed or is at least beginning to form in that the filament will conform to the inner shape of the capsule, resting against the inner walls. At this stage, the filament is moved back and forth (herein referred to as an “oscillating” movement) at a frequency of from zero up to 4 oscillations per second, preferably 0.2 to 1 oscillation per second. The oscillation can be made in different ways, i.e. longer movements back and forth of the filament (0.01 to 0.1 oscillations per second with about 1 to 5 cm of filament) or 0.1 to 1 oscillations per second with about 0.5 to 1 cm of filament.

These oscillations are performed during the insertion and removal of the filament, suitably between incremental steps of the introduction of the filament. It is also possible to provide a semi-automated system, by manual performing the feeding of filament with the option of having a mechanism that is controlling the process of feeding the filament backward or forward, or to provide a fully automated system, by coupling a motor to the filament for feeding and to have a control unit operating the motor so as to generate controlled feeding of the filament back and forth both during insertion and withdrawal.

Suitably a length of filament corresponding to the formation of three loops inside the lens capsule is introduced. However, it may be suffice with a single loop, and as many as up to 10 or 15 loops could be inserted, and even higher if the diameter of the filament is low (FIG. 8c-e). At present it is believed that optimal results will be achieved with a length of filament corresponding to a coil of 5 to 10 loops, i.e. 10 to 25 cm of filament.

When it is determined that residual material has been removed from the inner surface of the lens capsule in a satisfactory degree, the filament is retracted, and the lens capsule is cleaned from debris, suitably using an irrigation and aspiration device, that is a standard equipment in lens extraction surgery.

The results in terms of prevention of secondary cataract are very good, and the following examples serve to illustrate this.

FIGS. 9a and b shows performing the method from two directions in order to achieve a complete coverage of the interior lens capsule surface.

A bending of the tip was surprisingly found to add benefits to the invented device for performing the surgery. The bent tip improved the surgical use in patients with projecting eyebrows and nose, and to reach areas in the capsule that otherwise would be much more difficult. The bending is shown in FIG. 10 a, b, c.

The tip bending angle, as shown in FIG. 10 b and c, should be more than 5 degrees, preferably more than 20, but less than 90 degrees, preferably less than 45 degrees and most preferably about 30 degrees. The bending should preferably be smoothly rounded, shown in FIG. 10 c, but not excluding that it may be made in a sharp angle, as shown in FIG. 10 b.

The tip bending needs to be placed at a defined position on the tip, so that the filament loop is pointing either to the left or to the right, when the tip is bevelled up, as demonstrated in FIG. 10. In FIG. 10 a, the tip is bevelled up and out of the plane of the paper (which of course can not be seen in this illustration), and the loop is pointing to the right. In FIG. 10 b and c, the tip has been rotated 90 degrees counter-clockwise, so that the loop is pointing towards the observer. The opposite direction of the loop, i.e. pointing downwards, would also be okay, and facilitate the use of the device to reach left areas in the lens capsule.

Thus, the loop should be directed essentially perpendicularly from the plane of bending of the tip, although a deviation from a perpendicular orientation of the loop is acceptable. Such deviation should however not exceed 45 degrees in any direction, more than for very special surgical situations.

The filament may be fixed to the tip portion by winding one or several turns with the filament around the tip, and add glue over the winding, as shown in FIG. 11. This fixation method allows the filament to point in a desired angle of about 90 degrees (Angle X, in FIG. 11, and equivalent to Angle B, in FIGS. 2 and 3) from the tip after the last turn around the tip. The winding should preferably be made so that the last turn is closest to the distal tip end, so that the diameter of the loop could be made smallest for easy introduction through corneal micro-incisions of less than 1.5 mm, and preferably less than 1 mm. The winding should also preferably be made in just one layer, to minimize the total diameter of the tip and attachment.

The surface of the winding and the glue should be smooth, to avoid that the instrument gets stuck at the corneal incision. Another option is to make a knot of the filament to the tip. Preferably the used glue is UV-light hardening, for example DYMAX 1181M or 1191M. The surface of the tip at the attachment place may preferably be blasted for improved fixation to the tip.

The invention will now be described by non-limiting examples.

### EXAMPLES

### Example 1

### Experiments Using Different Filaments

Evaluation of lens-capsule dissection and shaving off lens epithelial cells from the inner capsular surface. Simulated lens extraction surgery in porcine cadaver eyes.

| | | Young's | | |
|---|---|---|---|---|
| | | Modulus | Diameter | |
| No | Filament material | GPa | mm | Outcome |
| 1 | Polyethylene | 0.2 | 0.5 | Loop forms in the lens capsule, dissects |
| | 0.1-1.2 GPa | | | the lens and the capsule, simultaneously |
| | | | | shaving off lens epithelial cells from the |
| | | | | inner capsular surface. |
| 2 | Polyethylene | 0.5 | 0.4 | Outcome as in Experiment no 1, and |
| | | | | easier lens - capsule dissection, and |
| | | | | more efficient cell removal |
| 3 | Polypropylene | 1.0 | 0.25 | Outcome as in Experiment no 2, and |
| | 1.0-2.0 GPa | | | formation of a couple of loops, and even |
| | | | | more efficient cell removal |
| 4 | Polyamid/nylon | 1.5 | 0.15 | Outcome as in Experiment no 3, and |
| | 1.2-3.0 GPa | | | formation of a several loops, and even |
| | | | | more efficient cell removal |
| 5 | Polyvinylidenefluoride | 2.0 | 0.14 | Outcome as in Experiment no 4, and |
| | 1.0-3.0 GPa | | | even more efficient cell removal |
| 6 | Reinforced | 2.5 | 0.12 | Outcome as in Experiment no 5, and |
| | Polyamid/nylon | | | formation of a coil of loops, and even |
| | 2.0-3.3 GPa | | | more efficient cell removal, and very |
| | | | | efficient lens - capsule dissection |
| 7 | Coated and | 3.0 | 0.12 | Outcome as in Experiment no 6. |
| | reinforced | | | |
| | polyamid/nylon | | | |
| 8 | Fluorocarbon | 3.0 | 0.12 | Outcome as in Experiment no 6. |
| | 2.5-3.0 GPa | | | |
| 9 | PMMA | 3.5 | 0.10 | Outcome as in Experiment no 6. |
| | 3.0-3.5 | | | |
| 10 | Reinforced | 5.0 | 0.10 | Outcome as in Experiment no 6. |
| | polyamid/nylon | | | |
| 11 | Reinforced | 10 | 0.08 | Outcome as in Experiment no 6. |
| | polyamid/nylon, | | | |
| | coated with Teflon | | | |
| 12 | Composite | 30 | 0.06 | Outcome as in Experiment no 1. |
| 13 | Composite | 50 | 0.04 | Outcome as in Experiment no 1. |
| 14 | Aramid (Kevlar ®) | 100 | 0.02 | Outcome as in Experiment no 1, and |
| | 59-124 GPa | | | more |
| 15 | NiTi alloy | 120 | 0.02 | Outcome as in Experiment no 1, and |
| | 70-120 GPa | | | more efficient cell removal |
| 16 | NiTi alloy - plastic | 80 | 0.03 | Outcome as in Experiment no 15. and |
| | composite | | | even more efficient cell removal |

The exemplified combinations of material and diameter in experiment no 5 to 11 are the most interesting, concerning functionality, efficacy, cost of material, and safety. These combinations are very promising in respect of low surface friction, high abrasion resistance and good stiffness/flexibility, high breakpoints which are important parameters to fulfil the criteria of an excellent product. Though, the exemplified combinations in experiments no 1 to 4 and 12 to 16 could also show similar results.

### Example 2

### Verification of the Invention by Showing the Different Parameters in Hand-Made Prototypes

The total length (Lt), outer diameter (OD), inner diameter (ID) as well as the choice of materials are presented. The total length Lt means the distance between the proximal and distal ends of the component, if not others are mentioned. For the filament the effective length (Le) is also presented, meaning the length of the filament introduced in the lens capsule that forms the loops and the coil of loops. The use of prototypes was evaluated by simulated lens extraction surgery in fresh porcine cadaver eyes received from a nearby slaughter-house. After the lens was extracted from the eye globe, cornea and iris were removed and the amount of remaining lens epithelial cells in the lens capsule was assessed by observation, and compared to the amount of cells assessed in control eyes, i.e. conventional lens extraction.

### Prototype I (Basic Concept)

Tube 2 stainless steel, L=100 mm, OD=0.81 mm, ID=0.50 mm
Tip portion 9 stainless steel, L=35 mm, OD=0.51 mm, ID=0.27 mm
Piston 5 stainless steel, L=100 mm, OD=0.48 mm, ID=0.30 mm
Filament 4 enforced nylon, Lt=250 mm, Le=120 mm, OD=0.15 mm
Fastening means 7 distal end of the filament unfixed, and the proximal end of the filament was unfixed to the proximal end of the piston.

Results and conclusions: The mechanism of the basic concept worked satisfactorily, and several loops were formed inside the lens capsule between the capsule and the lens. The amount of remaining lens epithelial cells in the lens capsule was lower compared to control eyes.

### Prototype II-VII (Design of Distal Filament End)

Specifications as for Prototype I, except for the design of the last part of the distal end of the filament. The purpose is to show that it is possible to avoid that the distal end of the filament is exiting from the lens capsular interior.
II tapered filament the last 10 mm to the distal end.
III closed loop with a diameter of about 5 mm, made of the last 15 mm of the filament
IV pre-shaped spiral
V patterned filament surface (combined with II, would also work with III and IV)
VI fixed to tip portion (or tube)
VII no distal filament end, i.e. two proximal ends and a distal loop

Results and conclusions: Similar as for Prototype I, with the benefit of improved control of the distal filament end. The distal end was prevented from leaving the capsule in a uncontrolled way.

### Prototype VIII-XII (Angle B of Fixation)

Specifications as for Prototype I, except changing the angle B of the fixation of filament to the tip at fastening means 7. The purpose was to show the influence of angle B on the properties of the device.

VIII Angle B: 0 degrees
IX Angle B: 45 degrees
X Angle B: 90 degrees
XI Angle B: 135 degrees
XII Angle B: 180 degrees

Results and conclusions: Similar as for Prototypes II to VII, with the benefit of different angels B, e.g. a large angle B (90-180 degrees) facilitates introduction through small corneal incisions and mini capsulorhexis, and a smaller angel B (0-90 degrees) improves the surgical performance within the lens capsule to shave off materials in difficult reachable areas of the lens capsular surface, so called “dead angle areas”.

### Prototypes XII-XVII (Length of Filament)

Specifications as for Prototype VI, except for different length of the filament, Lt and Le. The purpose is to show the influence of filament length.

### XIII Lt=300 mm, Le=180 mm

### XIV Lt=350 mm, Le=230 mm

### XV Lt=400 mm, Le=280 mm

### XVI Lt=500 mm, Le=380 mm

### XVII Lt=800 mm, Le=680 mm

Results and conclusions: Similar as for Prototype VI, with the benefit of a longer filament which improves shaving off materials from the inner capsular surface. The amount of loops formed in the capsule correlated to the effective length of the introduced filament Le. However, very long filaments, such as Le above 380 mm, increased the risk of entanglement between loops inside the capsule, but the capsule did remain intact. The final limit was set by that the space between the capsule and the lens was filled to capacity with loops of filament.

### Prototype XVII-XXII (Filament Materials)

The same specifications as for Prototype VI, except for different materials of the filament. The purpose is to show the influence of different filament materials. However, the Young's Modulus for the individual filaments were unknown. The approximate Young's Modulus (GPa) of the different materials according to standard tables was used instead.

XVII enforced polyamide (nylon) (approximately between 2 and 3 GPa)
XVIII enforced polypropylene (prolene) (approximately between 1 and 2 GPa)
XIX polyamide coated with silicon-PTFE (approximately between 3 and 4 GPa)
XX acrylic monofilament (suture material) (approximately around 1 GPa)
XXI a straw of hair (approximately 10 GPa and an OD of about 0.03 mm)
XII Stiff stainless steal wire (approximately 200 GPa and OD of about 0.20 mm)

Result and conclusion: Similar to Prototype VI, except the change in flexibility, stiffness and elasticity of the different materials. Stainless steal wire of the used dimension was too stiff to be used with risk to rupture the capsule. Materials with a Young Modulus between 1 and 10 GPa was shown to be okay, and that the dimension of the filament should be chosen in respect to choice of the material, i.e. a material with high Young's Modulus, such as 10 GPa and higher, would gain of having a smaller filament diameter. A nylon or prolene filament, with an approximate Young's Modulus between 1 GPa and 3 GPa, would be most suitable to have a diameter between 0.08 mm and 0.20 mm. More than 0.20 mm would probably limit the capacity to form a coil of multiple loops.

### Prototype XXIII-XIIV (Dual Mechanisms)

Purpose: To show the invention in respect of dual mechanisms.

XXIII Prototype similar to no I, but with double tubes 2 and 2′, and double pistons 9 and 9′, and temporarily fixed proximal filament ends by the locking member 10.
XXIV Prototype similar to no VII, combined with a tip portion with a rounding deflecting member 12 included.

Result and conclusions: Similar results as with Prototype VI, with the benefit of increased speed of introduction of filament in the capsular bag, and also the benefit of moving the filament back and forth from two directions, i.e. from either proximal filament end. If entanglement occurs, it could be removed easily by moving the filament in opposite direction.

### Example 3

### Verification of the Idea, by Showing the Different Varying Options to Use the Device During the Surgery

The use of prototype VI, described in Experiment 2, after capsulorhexis, but before lens extraction.
The use of prototype VI after lens extraction.
The use of prototype VI both before and after lens extraction.

Results and conclusions: Confirmed the invention that the device can be used before and/or after the lens extraction. When using it before lens extraction, as an option to hydro-dissection, admit a second use of the device after completed lens extraction as well.

### Example 4

### Verification of Invention in Rabbit Study

Purpose: To evaluate the invention for reduction of lens epithelial cells, and thereby prevent formation of secondary cataract. In this study, the use of the device was compared with conventional lens extraction in living rabbits concerning efficacy and safety. The rabbit model is well known for evaluation of ophthalmic surgical devices, such as IOLS, viscoelastic solutions and other surgical instruments.

Prototype: Equivalent as prototype I in Experiment no 1, but with a fixed distal end of the filament as in prototype XI, Experiment no 1. Only one kind of prototype was evaluated in this set up of animal experiment in order to keep the number of animals as low as possible due to animal ethics and costs of experiments.

Animals: 6 rabbits (New Zealand White, male)

Method: The use of the prototype was randomly chosen within each rabbit, one “prototype eye” i.e. lens extraction surgery including the use of the prototype, and one “control eye”, i.e. conventional lens extraction surgery without using the prototype. No artificial lenses were implanted in the eyes. Corneal thickness was measured by ultrasound at 2 and 7 weeks post surgery to detect any injuries to the cornea. After 7 weeks, the rabbits were sacrificed and the lens capsule with its content were extracted and measured by weight as a parameter of cell growth within the lens capsule.

Results and conclusions: A statistically significant difference (p<0.05) was observed regarding formation of secondary cataract between conventional surgery and the new improved method using the prototype. The new method did decrease the amount of secondary cataract/growth of lens epithelial cells. There was no difference between control and prototype eyes regarding pachymetry data, i.e. no signs of corneal injuries. No injuries to the capsule was observed.

### Example 5

### Experiment Made in Human Cadaver Eves

The purpose was to evaluate different prototypes of the invented device regarding safety and surgical performance in human donor eyes as a prerequisite of clinical studies and regulatory requirements.

By using the device, the complete lens is loosened from the capsule, simultaneously as cortex removal and shaving off epithelial cells are achieved. A central capsulorhexis with a diameter between 4 and 6 mm were used in the experiment. Other types, sizes and placements of the capsulorhexis may give other kind of results and conclusions.

Previous studies of the device have been made in rabbit eyes for studying prevention of secondary cataract (posterior capsular opacification, PCO) and surgical performance by experienced surgeons. It has also been suggested that a completely removed lens cortex may improve IOL positioning in the capsular bag.

In the present study, an experienced surgeon made the surgery and the evaluation, including histopathology of remaining lens epithelial cells in the capsular bag. He used the device for the first time, and was learning during the tests. Prototypes with filament dimensions 5/0 (0.14 mm in diameter) and 6/0 (0.10 mm) were evaluated and compared to hydro dissection.

The human donor eyes were used between 3 and 5 days post enucleation. The physiological qualities of the eyes deteriorate over time, such as the zonula ciliaris and the attachment of the lens epithelial cells to the capsular bag, and constitute potential sources of error.

The following results and conclusions were made. The results and the conclusions may differ at another surgical set up, such as in eyes of living patients:
The surgical performance with prototypes of the invented device was found to be safe in surgery of the human donor eyes. However, the device should not be used at a pre-existing zonulolysis, a capsular tear or a non-continuous capsulorhexis.
The preferred surgical technique was to use the invented device from two opposite corneal incisions at three different positions from each incision.
The filament dimension 6/0 was found to be more suitable than 5/0, since the larger diameter made the loop stiffer. The stiff loop lifted the iris which in turn may cause iris depigmentation. Approximately 3 cm of the filament was introduced into the capsule, i.e. small single loops of the filament were preferred to be used, rather than multiple loops.
The prototypes required 1.5 mm wide corneal incisions for being introduced. A smoother surface and a smaller dimension of the device's tip portion would facilitate introducing the device through smaller corneal incisions.
The use of the invented device took approximately 3 minutes. The time may be shortened by improved instrumental design and extended surgical training. In addition, the invented device would reduce time and need of hydro dissection, cortex removal and capsular polishing by irrigation/aspiration (I/A).
Approximately 5-20% of lens cortex remained at the capsular periphery, when estimated in the surgical microscope. A remaining monolayer of lens epithelial cells at the capsular periphery was seen by histopathology, but no evident multi layers of cells. However, the deterioration of the eye tissues over time most likely affected the test results.

No conclusion could be made regarding the differences between the invented device and hydro dissection concerning removal of lens epithelial cells. The number of eyes was too small, and the human donor eyes were found unsuitable due to the deterioration over time. A specific study should be designed to compare the invented device and hydro dissection, preferably in living rabbit eyes.

1. A device for removing undesired tissue such as residual tissue, epithelial cells and/or other undesired material(s) from an inner surface of a lens capsule of an eye, comprising an elongated body (2) having a proximal and a distal end and at least one central lumen extending between said ends; a flexible shaving filament (4) movably provided in said lumen so as to be insertable into the lens capsule; wherein said filament has an overall stiffness such that it will be able to conform to an inner surface of a lens capsule when inserted into said capsule, and also to enable shaving off of material from said inner surface.

2. The device as claimed in claim 1, wherein the actual length of introduced filament in a fully projected state inside a lens capsule amounts to at least 1 cm, preferably more than 2 cm, more preferably to more than 2.5 cm, even better more than 3 cm, but preferably less than 100 cm, more preferably less than 50 cm, preferably less than 35 cm, and suitably less than 25 cm.

3. The device as claimed in claim 2, wherein a single loop is to be formed wherein the filament is between 1 cm and 6 cm, preferably more than 2 cm, or even better more than 3 cm, but preferably less than 5 cm, and suitably less than 4.5 cm. An optimal length would be 3 to 4 cm.

4. The device as claimed in claim 1, wherein the flexible filament (4) has a length from the distal end of said body (2) to the tip of the filament (4) when it is fully introduced into the lens capsule and/or in a fully protruded state, said length being at least 1.2 times the inner circumference of a lens capsule; or at least 25 mm for a human adult lens; or a length exceeding the circumference of the lens capsule.

5. The device as claimed in claim 1, wherein the flexible filament (4) has a length from the distal end of said body (2) to the tip of the filament (4) such that it forms at least one loop, preferably forms a couple of loops and even more preferably forms a coil of several to multiple loops, when it is fully introduced into the lens capsule and/or in a fully protruded state.

6. The device as claimed in claim 1, wherein the flexible filament (4) has a diameter of 0.02 mm-0.50 mm, preferably more than 0.05 mm, still more preferable greater than 0.10 mm, but more preferred less than 0.25 mm, and even more preferred less than 0.15 mm.

7. The device as claimed in claim 1, wherein the flexible filament (4) has a Young's modulus in the range of more than 0.5 GPa, preferably more than 1 GPa, still more preferable greater than 2 GPa, but less than 200 GPA, more preferred less than 100 GPa, and even more preferred less than 30 GPa, suitably less than 15 GPa.

8. A device as claimed in claim 1, comprising means for feeding said flexible filament through said lumen to protrude out from said distal end.

9. The device as claimed in claim 1, wherein the inner diameter of said central lumen of the hollow tube (2) is not more than 0.40 mm larger than the diameter of the filament, preferably not more than 0.20 mm larger, most preferred not more than 0.05 mm larger than the diameter of the filament.

10. The device as claimed in claim 1, wherein the cross-section of the filament is circular.

11. The device as claimed in claim 1, wherein the cross-section of the filament is elliptic.

12. The device as claimed in claim 1, wherein the cross-section of the filament is essentially flat and ribbon-like.

13. The device as claimed in claim 1, wherein multiple loops are to be formed, wherein the actual length of introduced filament in a fully projected state inside a lens capsule amounts to between 5 cm and 50 cm, or even up to 100 cm, preferably more than 10 cm, but preferably less than 35 cm, and suitably less than 25 cm. An optimal length would be 15 to 20 cm.

14. The device as claimed in claim 1, wherein the diameter of the filament is between 0.02 mm to 0.50 mm, more preferably between 0.05 and 0.25 mm and most preferably between 0.10 to 0.15 mm.

15. The device as claimed in claim 1, wherein one end of the filament is fixed to the distal end of the device.

16. The device as claimed in claim 1, wherein the device comprises two lumen and wherein the filament runs through both lumen to form a closed loop at the distal end.

17. The device as claimed in claim 1, further comprising a tip portion having a smaller diameter than the elongated hollow body.

18. The device as claimed in claim 17, wherein the tip portion has an outer diameter of 0.2 to 1.2 mm.

19. The device as claimed in claim 1, wherein a tip portion of the elongated body is bent to form an angle of more than 5 degrees, preferably more than 20, but less than 90 degrees, preferably less than 45 degrees and most preferably about 30 degrees.

20. The device as claimed in claim 19, wherein the angle of the bend is sharp.

21. The device as claimed in claim 19, wherein the bend is smooth, i.e. extends over a section of the elongated body.

22. The device as claimed in claim 1, wherein the filament is attached to the distal end of the elongated body by winding the filament around the elongated body.

23. The device as claimed in claim 22, wherein the filament is wound around the tip in at least 0.5 turn, preferably about 2.5 turns.

24. The device as claimed in claim 23, wherein the filament is secured to the elongated body by applying glue, suitably a UV curing glue.

25. The device as claimed in claim 1, wherein the overall stiffness S of the filament is more than 1 Newton, preferably more than 3 Newton, still more preferable greater than 4 Newton, and suitable more than 6 Newton, but less than 140 Newton, more preferred less than 70 Newton, and even more preferred less than 35 Newton, and suitable less than 20 Newton, as measured by the method disclosed in the specification.

26. A device for removing undesired tissue such as residual tissue, epithelial cells and/or other undesired material (s) from an inner surface of a lens capsule of an eye, comprising an elongated body (2) having a proximal and a distal end and at least one central lumen extending between said ends; a flexible shaving filament (4) movably provided in said lumen; wherein one end of said filament is attached to the distal end of said elongated body, such that when the filament is moved inside the lumen to extend out from the lumen at least one loop will form.

27. The device as claimed in claim 26, said filament having an overall stiffness such that it can be introduced into the lens capsule and also will be able to conform to an inner surface of a lens capsule when inserted into said capsule, and also to enable shaving off of material from said inner surface.

28. The device as claimed in claim 1, wherein the filament has a free end said free end being insertable into the lens capsule, and wherein the stiffness (S) of the filament varies from between more than 1 Newton, preferably more than 3 Newton, still more preferable greater than 4 Newton, and suitable more than 6 Newton, but less than 140 Newton, more preferred less than 70 Newton, and even more preferred less than 35 Newton, and suitable less than 20 Newton, as measured by the method disclosed in the specification, over the majority of its length, and decreases towards the free end, preferably down to near 0 N at the very distal end of said filament.

29. A method of removing undesired tissue such as residual tissue, epithelial cells and/or other undesired material (s) from the inner surface of a lens capsule of an eye of a mammal, comprising the steps: introducing a flexible shaving filament into the lens capsule, comprising feeding a length of said filament into the lens capsule such that the filament is brought into contact with an inner surface of the lens capsule and conforms to said inner surface of said lens capsule when inserted into said capsule, and also to enable shaving off of material from said inner surface; and moving the filament back and forth to cause an oscillating movement of said loop(s) so as to shave off said epithelial cells/undesired tissue/undesired material (s) from said inner surface.

30. The method as claimed in claim 29, wherein the feeding of said filament is incremental, and wherein the moving of the filament moved back and forth is done between increments.

31. The method as claimed in claim 29, further comprising alternating feeding and oscillating movements.

32. The method as claimed in claim 29, wherein the actual length of introduced filament in a fully projected state inside a lens capsule amounts to at least 1 cm, preferably more than 2 cm, more preferably to more than 2.5 cm, even better more than 3 cm, but preferably less than 100 cm, more preferably less than 50 cm, preferably less than 35 cm, and suitably less than 25 cm.

33. The method as claimed in claim 29, wherein the length of filament is 15-20 cm.

34. The method as claimed in claim 29 wherein the filament is inserted into the lens capsule through a hollow elongated body.

35. The method as claimed in claim 29, wherein the filament forms at least one loop, preferably a couple of loops, even more preferably a coil of several loops.
